(19) United States
(12) Patent Application Publication
(10) Pub. No.: US 2005/0192290 A1
(43) Pub. Date: Sep. 1, 2005

(54) Treatment of behavioral disorders

(76) Inventor: Isaac Melamed, Englewood, CO (US)

Correspondence Address: TIMOTHY J MARTIN, PC, 9250 W 5TH AVENUE, SUITE 200, LAKEWOOD, CO, 80226, US

(21) Appl. No.: 11/036,182

(22) Filed: Jan. 13, 2005

Related U.S. Application Data
(60) Provisional application No. 60/536,458, filed on Jan. 13, 2004.

(51) Int. Cl. A61K 31/495; A61K 31/473

(52) U.S. Cl. 514/255.04; 514/290

(57) ABSTRACT

The present invention relates to a method for treating a behavior disorder comprising the administration of a therapeutically effective amount of antihistamine, such as ceterizine, fexofenadine; loratadine, and desloratadine. The behavioral disorders may include ADHD, anxieity, depression, and autism. The method may include the administration of the antihistamine in combination with a stimulant medication, such as methylphenidate, thereby to achieve a synergistic effect. In any event, the amount of antihistamine and/or stimulant is effective to downregulate neurotrophic factors such as nerve growth factor or CD40. The invention is also directed to a method of preventing the onset of behavior disorders in patients presenting with symptoms of allergic rhinitis.

### FIELD OF THE INVENTION

The present invention generally relates to methods for the management and/or pharmacological treatment of certain behavioral disorders. More specifically, the present invention concerns the treatment of symptoms related to attention deficit/hyperactivity disorder (ADHD), anxiety, depression and autism through the administration of a treatment, or a combination of treatments, effective to regulate neurotrophic factors. The present invention also concerns a method for treating comorbid allergic rhinitis and behavioral disorders either with a single medication or by the synergistic effect created by a plurality of medications.

### BACKGROUND OF THE INVENTION

Allergic rhinitis afflicts millions of people all over the world. The associated health costs and economic loss due to missed workdays are significant. Allergic rhinitis also significantly impacts the health of children. Statistics confirm that up to 30% of all children are afflicted with allergic rhinitis. Children who attend school, while symptomatic, are often described as apathetic, absent-minded, poorly focused, forgetful, and disinterested in both educational and social activities. Evidence further supports that a child's cognitive functioning can be impaired by these allergy symptoms. As a result, allergic rhinitis can diminish a child's ability to learn, concentrate, and interact socially.

Various treatments of allergic rhinitis and allergic symptoms have adverse side effects. For example, some treatments might cause a child to be inattentive or occasionally overactive. In addition, sedation and reduced alertness, as well as impairment of cognitive functions and psychomotor performance, have long been associated with sedative antihistamines. The use of ceterizine, however, has shown positive results for managing allergic rhinitis with few side effects. Ceterizine, also known in the industry under the trademark Zyrtec® manufactured by Pfizer/UCB, Inc., a Delaware corporation, is a recently developed, long-acting (once-a-day), non-sedating, H1 receptor antagonist with proven antihistamine activity in the treatment of seasonal allergic rhinitis.

Children diagnosed with allergic rhinitis have learning and focusing problems, which are two of the most common symptoms of children with a behavioral disorder known as attention deficit/hyperactivity disorder (ADHD). Similarly, ADHD children are often reported to display signs of allergies to various substances and/or atopic symptoms (i.e., atopic eczema, hay fever or asthma). The association between allergic rhinitis and ADHD has been often touted and the clinical overlap between allergic rhinitis and ADHD is evident. In fact, research suggests that children with ADHD and children with allergic diseases may share a common biological background.

Children who have ADHD represent a very diverse heterogeneous population and exhibit a broad spectrum of symptom severity, as well as a wide range of associated diagnoses. Some of the symptoms associated with ADHD include inattention, hyperactivity, and impulsivity. In addition, other conditions such as anxiety, depression, and autism may also be present in those afflicted by ADHD. The usual course of treatment for children suffering from ADHD may include such medications as methylphenidate, also known in the industry as Ritalin SR®, manufactured by Ciba-Geigy Corporation, a division of Novartis, Inc. Methylphenidates are stimulants that decrease impulsivity and hyperactivity and increase attention.

Despite the various beneficial effects related to the treatment of ADHD with methylphenidates, there are a variety of side effects associated with its use including loss of appetite, insomnia, headaches, stomachaches, drowsiness, hyperactivity, blood pressure and pulse changes, and cardiac arrhythmia. Even more unsettling is that it is currently unknown whether risks are involved with long-term use of methylphenidates.

Accordingly, there remains a need to provide a treatment for ADHD that is safe and that has less severe side effects than those associated with methylphenidates. There is also a need to provide an equally safe treatment for the associated conditions of anxiety, depression, and autism. Further, due to the overlap of individuals presenting with both allergic rhinitis and ADHD, there is a further need to provide treatment for ADHD, anxiety, depression, and autism that does not counteract with the treatment of allergic rhinitis. The present invention is directed to meeting these needs.

### SUMMARY OF THE INVENTION

An object of the present invention is to provide a new and useful treatment for ADHD;

Another object of the present invention is to provide a treatment for ADHD that does not necessarily require a stimulant medication such as methylphenidate, but which alternatively can be used to improve response to treatment;

Still another object of the present invention is to provide a new use for an antihistamine;

Yet another object of the present invention is to provide a concomitant treatment for allergic rhinitis and ADHD;

Still, a further object of the present invention is to provide a method for treating a patient exhibiting symptoms of ADHD; and

Another object of the present invention is to provide a new treatment for depression and autism.

In accordance with these objectives, then, the present invention broadly concerns new method of treating a behavioral disorder comprising the administration of a therapeutically effect amount of antihistamine. The antihistamine may be any suitable antihistamine, such as ceterizine, fexofenadine, loratadine, or desloratadine, that is administered in an amount sufficient to ameliorate the behavioral disorder. More particularly, the antihistamine is administered in an amount sufficient to downregulate neurotrophic factors, and specifically nerve growth factor (NGF) and CD40. The antihistamine may be used to treat ADHD, anxiety, depression, and autism.

The present invention also contemplates the administration of both an antihistamine and a stimulant medication for the treatment of behavioral disorders. The antihistamine and the stimulant medication are administered in a respective first and second therapeutically effective amount sufficient to create a synergistic effect for the down regulation of the neurotrophic factors thereby regulate the behavioral disorder. The stimulant medication can specifically be methylphenidate.

The present invention is also directed to a method of regulating neurotrophic factors with an antihistamine either alone or in combination with a stimulant medication. Additionally, the present invention is directed to a method of simultaneously treating allergic rhinitis and a behavioral disorder either with an antihistamine alone or in combination with a stimulant medication.

The present invention further contemplates a method of preventing the onset of behavioral disorders in a patient presenting with a symptom, or symptoms, associated with allergic rhinitis, comprising, administering a therapeutically effective amount of an antihistamine sufficient to downregulate neurotrophic factors.

These and other objects of the present invention will become more readily appreciated and understood from a consideration of the following detailed description of the exemplary embodiments of the present invention when taken together with the accompanying drawings, in which:

### BRIEF DESCRIPTION OF THE DRAWINGS

FIG. 1 is a graph showing Connors hyperactivity t-scores by group;

FIG. 2 is a graph showing Connors ADHD t-scores by group;

FIG. 3 is a graph showing Connors inattention t-scores by group; and

FIG. 4 is graph showing Connors optional t-scores by group.

### DETAILED DESCRIPTION OF THE EXEMPLARY EMBODIEMENTS

The present invention concerns the treatment of behavioral disorders and comorbid allergic rhinitis. The present invention also concerns the treatment of ADHD and various other behavioral disorders that may also be present including anxiety, depression, and autism. More particularly, the present invention concerns the use of an antihistamine, either alone or in combination with other medicaments, for the regulation or management of neurotrophic factors, specifically, the downregulation both nerve growth factor (NGF) and CD40.

As noted in the background portion of this disclosure, there is an apparent association between allergic rhinitis and ADHD. In effort to better understand this association, the inventor of the present invention developed studies, discussed in greater detail below, to determine whether an overlap exists between the allergy/immune system and the nervous system. The results of these studies proved the importance of the nervous system and neurotrophic factors in the regulation of the immune system. Such regulation could be mediated by neurosubstances released into the lymphoid microenvironment. Indeed, the studies indicated that various neurotransmitters and hormones affect proliferation and differentiation of cells of the immune system, and their respective receptors have been demonstrated on lymphoid cells.

Before developing a new treatment and/or management of ADHD related behavior parameters, it was first necessary to understand this linkage or association between the allergy/immune system and the central nervous system. To this end, the inventor studied the neurotrophic protein nerve growth factor (NGF) and another neurotrophic protein called CD40, which is part of the super-family of NGF. More particularly, CD40 is a 50 kDa member of the tumor necrosis factor receptor (TNFR) family of proteins that also includes TNF-R1 and nerve growth factor receptor (NGFR). Along with NGF, CD40 is involved in many other signaling pathways and significantly contributes to the inflammatory process. These two proteins have been shown to be extensively involved in the etiology of autism.

The study of these neurotrophic proteins, which is discussed in more detail below, indicated that both NGF and CD40 play a significant role in the inflammatory process and provide a cross-linkage between the immune system and the central nervous system. Based upon this cross-linkage, it was further discovered that due to the neuroimmune linkage of NGF and CD40, the inflammatory process is the connection between the mental disorders of ADHD, depression, and autism. From this finding, the inventor later discovered that antihistamines, such as Zyrtec®, have a beneficial effect on the parameters of ADHD. Furthermore, antihistamines, such as Zyrtec®, have a significant effect on the regulation of NGF and CD40 indicating the possibility of providing an effective treatment for both depression and autism.

I. Experimentation

In general, two studies were conducted to better understand the association between the allergy/immune system and the central nervous system. STUDY A, tested the effects of ceterizine (namely Zyrtec®), ceterizine and methylphenidate (namely Ritalin SR®), and methylphenidate, versus placebo on attention, memory, and behavior in children with ADHD and allergic rhinitis. STUDY B assessed NGF response to ceterizine and methylphenidate treatment in children with ADHD and allergic rhinitis.

A. Test Subjects

For both studies, 220 patients were screened, 60 randomized, and 38 completed the study. (9 female, 29 male). The individuals chosen to be a part of these studies had the following characteristics:
1) Male or female patients between 8-18 years of age.
2) History and diagnosis of seasonal allergic rhinitis to a prevalent allergen.
3) Documented seasonal allergy to a prevalent allergen (grass or tree) as confirmed by a recognized skin test: prick or intradermal (I.D.) (Prick wheal >3 mm over the negative control; intradermal wheal >5 m over the negative control).
4) Seasonal allergic rhinitis to a prevalent allergen of such severity that it required pharmacological therapy each year for the last 2 consecutive years (including the present year). Every subject was diagnosed previously with ADHD and was on pharmacological therapy with a stimulant medication.

B. Four Treatment Groups

All subjects were randomized into one of four treatment groups as follows:
1) Ceterizine alone: 10 mg po qam for 2 weeks;
2) Combination of ceterizine and methylphenidate: 10 mg po qam+Ritalin SR® (20 mg if <65 kg; 40 mg if ≧65 kg) po qam for 2 weeks;
3) Methylphenidate alone: (20 mg if <65 kg; 40 mg if ≧65 kg) po qam for 2 weeks; and
4) Placebo.

C. Measures Assessed

Every subject was assessed for the following measures:

Allergic Rhinitis Measures
1) Rhinoconjunctivitis Symptoms
2) Total Symptom Severity Complex, (TSSC)
3) Adolescent Rhinoconjunctivitis Quality of Life Questionnaire, (RQLQ)

ADHD Measures:
1) Child Behavior Checklist, (CBCL)
2) Child Symptom Inventory, (CSI)
3) Multi-dimensional Assessment of Anxiety in Children, (MASC)
4) Children's Depression Inventory, (CDI)
5) Conners' Rating Scale—Revised, (CRS)
6) Conners' Continuous Performance Task, (CPT)
7) California Verbal Learning Test, (CVLT)
8) NGF Assay
   II. Results

A. Study A: Test Results for Allergic Rhinitis

Based on the findings, with respect to the alleviation of Rhinoconjunctivitis symptoms and RQLQ, this study suggested that the methylphenidate had a superior effect compared to ceterizine on rhinoconjuctivitis quality of life scores and RQLQ nasal symptoms. The combination of ceterizine and methylphenidate yielded a better impact on rhinoconjuctivitis quality of life scores and RQLQ nasal symptoms compared to use of the drugs individually. As to the RQLQ emotional symptoms, the change from baseline was similar in the ceterizine and the methylphenidate group.

B. Study A: Test Results for ADHD

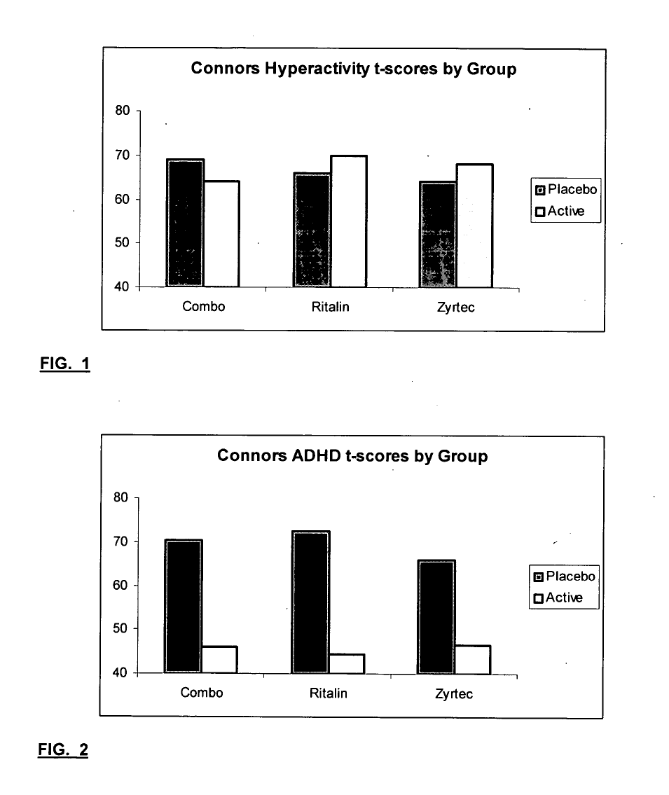

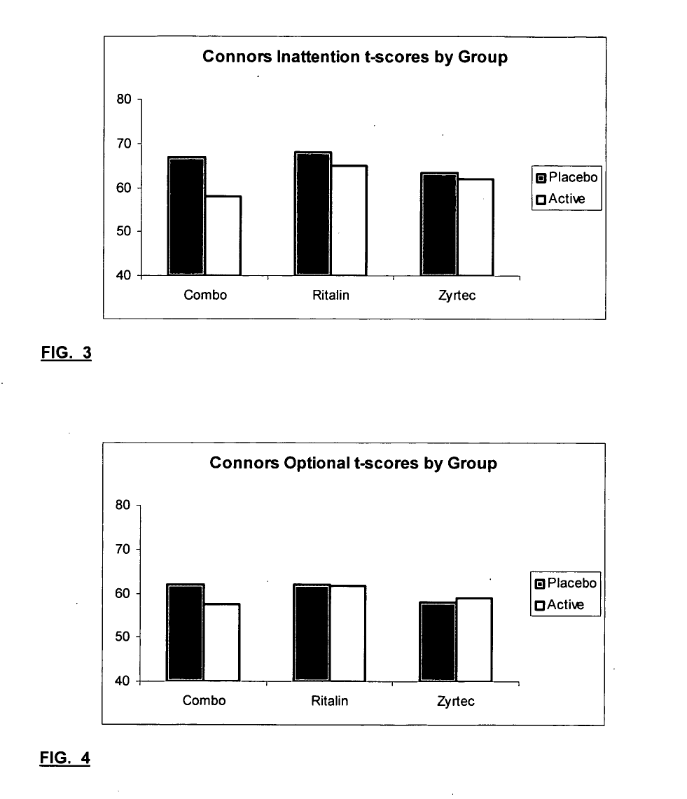

As shown by the graph in FIG. 1, the evaluation of the ADHD parameters revealed that the combination therapy had a better effect on hyperactivity t-scores (FIG. 1) while no difference was noted between the methylphenidate and the ceterizine groups. The parental Conner's report showed that hyperactivity (FIG. 2), ADHD, inattention (FIG. 3) and oppositional scores (FIG. 4) improved the most in the combined drug group, while no difference was recorded between the ceterizine and the methylphenidate group. As shown in FIGS. 1-4, there is very little difference in the test results when the patient was treated with ceterizine alone or with methylphenidate alone. However, as shown in the Figures, improved patient results were achieved when the patient was treated with a combination of ceterizine and methylphenidate.

C. Study B: Test Results For NGF

NGF was measured by ELISA (Enzyme-Linked Immunosorbent Assay) and the three groups analyzed. NGF was the highest when on placebo. It was downregulated to the same extent in the ceterizine and the methylphenidate groups. An even greater downregulation was documented in the combined group. Accordingly, children with ADHD and allergies have high expression of serum NGF. Ceterizine and methylphenidate taken individually suppress NGF to the same extent. However, when the two are taken together, the suppression of NGF is significantly increased over independent dosage.

III. Discussion of Results

Based upon the foregoing studies, the findings show that NGF plays a major role in the communication between the nervous system and the immune system, principally in regards to allergic response and ADHD. More particularly, the immune system responds to allergic reactions by increasing levels of NGF. Increased levels of NGF affects the central nervous system thereby initiating the process that commences behavioral disorders such as ADHD, anxiety, depression, and autism.

The studies relating to CD40 suggest that the functions of the TNFR family are quite divergent. For example, Fas and TNFR induce apoptosis following stimulation, whereas CD40 and NGFR rescue cells from apoptosis. In addition to its expression on B-cells, CD40 is also found in dentritic cells, activated macrophages, epithelial cells, and several tumor cell lines. Along with NGF, the studies indicate that CD40 is involved in many other signaling pathways and significantly contributes to the inflammatory process.

These findings suggest that there exists a linkage between the nervous system and the immune system. Due to this linkage, and based upon the conclusions drawn from the studies, allergies appear to play an etiological role in the small subgroup of children who suffer from ADHD. Additionally, the results suggest that the inflammatory process, due to the neroimmune linkage of NGF and CD40, is connected to the behavioral disorders of ADHD, anxiety, depression, and autisim. The studies further suggest that NGF and CD40 play a significant role and are the critical link in the pathogenesis of autism and each is extensively involved in the etiology of autism.

Based upon the foregoing, then, antihistamines, such as ceterizine, will have a significant effect on the regulation of NGF and CD40. As such, a therapeutically effective amount of antihistamines can be used to treat a patient presenting with one or more behavioral disorders such as ADHD parameters, anxiety, depression, and autism. Accordingly, antihistamines can be used as a first course of treatment rather than a stimulant medication, in an effort to manage these behavioral disorders. Treatment of the behavioral disorders, with antihistamines, provides the patient with an attractive alternative to treatment with methylphenidates because, as discussed in the background portion of this disclosure, the side effects associated therewith are typically fewer and less severe.

Alternatively, according to the present invention, a synergistic effect created by administering a first therapeutically effective amount of antihistamines and a second therapeutically effective amount of methylphenidates can be used to manage or regulate one or more of these behavioral disorders, namely ADHD parameters, anxiety, depression, and autism. As should be appreciated, the combination of the antihistamines and the methylphenidates would result in a lesser dosage of the methylphenidate than if using the methylphenidate alone, and thus would still be advantageous to reducing the number or severity of the side effects associated with methylphenidates.

Further, as should be appreciated, antihistamines other than ceterizine (Zyrtec®) may also be used to manage the behavioral disorders. For example, antihistamines, such fexofenadine, loratadine, and desloratadine may be used. Fexofenadine is commonly known in the industry under the trademark Allegra®, (manufactured by Hoechst Marion Roussel, a Delaware corporation). Loratadine and desloratadine are commonly known in the industry under the trademarks and Claratin® and Clarinex®, respectively (both of which are manufactured by Schering Plough, a New Jersey corporation).

Finally, as contemplated, the regulation of NGF and CD40 can assist in the prevention of the development of the behavioral disorders and particularly in those individuals presenting with allergic rhinits. Use of antihistamines to regulate NGF and CD40 provides a reduction of the inflammation associated with allergic rhinitis. This reduction in inflammation has an important effect of preventing the cascade of immune response that leads to the effects on the nervous system by communication via NGF and CD40. If left untreated, the eventual process of this inflammation precipitates ADHD, depression and eventually autism.

Accordingly, the present invention has been described with some degree of particularity directed to the exemplary embodiments of the present invention. It should be appreciated, though, that the present invention is defined by the following claims construed in light of the prior art so that modifications or changes may be made to the exemplary embodiments of the present invention without departing from the inventive concepts contained herein.

1. A method for treating a behavioral disorder, comprising administering a therapeutically effective amount of an antihistamine.

2. A method according to claim 1 wherein said antihistamine is selected from the group consisting of ceterizine, fexofenadine, loratadine, and desloratadine.

3. A method according to claim 1 wherein said antihistamine is ceterizine.

4. A method according to claim 1 wherein said therapeutically effective amount is sufficient to downregulate neurotrophic factors.

5. A method according to claim 4 wherein said neurotrophic factors are selected from the group consisting of nerve growth factor (NGF) and CD40.

6. A method according to claim 1 wherein the behavioral disorder is selected from the group consisting of ADHD, anxiety, depression, and autism.

7. A method according to claim 1 wherein the behavioral disorder is autism.

8. A method for treating a behavioral disorder, comprising
(A) administering a first therapeutically effective amount of an antihistamine in; and
(B) administering a second therapeutically effective amount of a stimulant medication, whereby said first and second therapeutically effective amounts create a synergistic effect.

9. A method according to claim 8 wherein said antihistamine is selected from the group consisting of ceterizine, fexofenadine, loratadine, and desloratadine.

10. A method according to claim 8 wherein said antihistamine is ceterizine.

11. A method according to claim 8 wherein said stimulant medication is methylphenidate.

12. A method according to claim 8 wherein said synergistic effect downregulates neurotrophic factors.

13. A method according to claim 12 wherein said neurotrophic factors are selected from the group consisting of nerve growth factor (NGF) and CD40.

14. A method according to claim 8 wherein the behavioral disorder is selected from the group consisting of ADHD, anxiety, depression, and autism.

15. A method of regulating neurotrophic factors comprising, administering a first therapeutically effective amount of an antihistamine sufficient to ameliorate symptoms associated with ADHD.

16. A method according to claim 15 wherein said antihistamine is selected from the group consisting of ceterizine, fexofenadine, loratadine, and desloratadine.

17. A method according to claim 15 wherein said antihistamine is ceterizine.

18. A method according to claim 15 wherein the neurotrophic factors are selected from the group consisting of nerve growth factor (NGF) and CD40.

19. A method according to claim 18 wherein said therapeutically effective amount is sufficient to downregulate NGF.

20. A method according to claim 18 wherein said therapeutically effective amount is sufficient to downregulate CD40.

21. A method according to claim 15 including the step of administering a second therapeutically effective amount of a stimulant medication whereby said first and said second therapeutically effective amounts create a synergistic effect.

22. A method for treating allergic rhinitis and a behavioral disorder comprising, administering a first therapeutically effective amount of an antihistamine.

23. A method according to claim 22 including the step of administering a second therapeutically effective amount of a stimulant medication whereby said first and said second therapeutically effective amounts create a synergistic effect.

24. A method according to claim 23 wherein said antihistamine is selected from the group consisting of ceterizine, fexofenadine, loratadine, and desloratadine and wherein said stimulant medication is methylphenidate.

25. A method according to claim 22 wherein said antihistamine is ceterizine.

26. A method according to claim 22 wherein said first therapeutically effective amount is sufficient to downregulate selected neurotrophic factors.

27. A method according to claim 26 wherein said neurotrophic factors are selected from the group consisting of nerve growth factor (NGF) and CD40.

28. A method according to claim 22 wherein the behavioral disorder is selected from the group consisting of ADHD, anxiety, depression, and autism.

29. A method of preventing the onset of behavioral disorders in a patient presenting with a symptom associated with allergic rhinitis, comprising, administering a therapeutically effective amount of an antihistamine sufficient to down-regulate neurotrophic factors.
